(19) United States
(12) Patent Application Publication
(10) Pub. No.: US 2005/0053676 A1
(43) Pub. Date: Mar. 10, 2005

(54) Powdered composition for use as laxative

(75) Inventors: Martin Schata, Dusseldorf (DE); Christian Pullen, Koln (DE)

Correspondence Address: CONNOLLY BOVE LODGE & HUTZ LLP, SUITE 800, 1990 M STREET NW, WASHINGTON, DC, 20036-3425, US

(73) Assignee: Madaus AG, Koln (DE)

(21) Appl. No.: 10/805,216

(22) Filed: Mar. 22, 2004

(30) Foreign Application Priority Data
Sep. 5, 2003 (DE) 103 41 403.7
Oct. 4, 2003 (DE) 103 46 083.7

(51) Int. Cl. A61K 31/715; A61K 35/78; A61K 33/00

(52) U.S. Cl. 424/724; 424/738; 514/54

(57) ABSTRACT

Described is a laxative on the basis of a pulverized composition, which contains in addition to plantago seed (Plantaginis ovatae semen) and/or plantago seed husks at least an anthranoid compound having a purgative effect, at least a sennoside, preferably in the form of anthranoid or sennoside-containing plants (or constituent parts thereof), in addition to at least one polygalactomannan-based polysaccharide or a derivative thereof. The polygalactomannan-based polysaccharide or a derivative thereof acts on the one hand as a (co)stabilizer in an aqueous suspension of the pulverized composition, and on the other hand also has, in particular due to its swelling capability, a synergistic effect together with the other components of the composition, wherein it supports the purgative action and facilitates at the same time the workability of the composition according to the invention, which is processed into a fine powder that can be easily dispersed in water.

The present invention relates to a powdered composition for use as a laxative (purgative agent, aperient). In particular, the present invention relates to a powdered composition of plantago seeds and/or plantago seed husks, as well as anthranoid compounds, in particular sennosides, preferably in the form of parts of plants or plant constituents containing anthranoids or sennosides, as well as to the use of this composition as a laxative (purgative agent, aperients).

Laxatives—also referred to synonymously as “aperients” or “purgative agents”, are available in many physical forms, including also numerous vegetable laxatives.

It is known for example that plantago seed (Plantaginis ovatae Semen or Semen plantaginis ovatae, referred to synonymously also as Indian psyllium, blond psyllium, Ispaghula or Semen Ispaghulae) or its seed husks (plantago seed husks, Plantaginis ovatae seminis integumentum) can be used in medications employed for regulation of bowel functions. Plantago seed possesses a significant swelling capability and induces a physical expansion and excitation in the sensitive receptors of the intestinal wall. Also, according to a known method (compare DE-PS 11 03 520), the seeds are finely ground, premixed with water to create a viscous paste and dried in the bundle form which is broken up and eventually sugar coated.

The action of the senna plant (senna plant, Cassia senna L. and Casia angustifolia Vahl), in particular of its fruits (Fructus Sennae, synonymously referred to also as senna husks or senna follicles) (for example Alexandrian senna fruits=Sennae fracture acutifoliae and/or Tinnevelly senna fruits=Sennae fructus angustifoliae) or its fruit pods such as its leaflets (Folia Sennae), is also known as a vegetable laxative means.

Also known are laxatives which combine both of the above-named active principles, for example laxatives in which the physical action of the plantago seed is reinforced by the pharmacologically stimulating active principle of sennosides, the content components of the senna fruits, in order to attain a superior total action.

It is further also known that mixtures can be manufactured from ground plantago seed and senna fruits as simple mixtures, wherein the ingredients are present next to each other. With this type of mixtures, however, an optimal adjustment of the free-flowing capability and suspending capability in water cannot be achieved. On the other hand, this type of adjustment is desirable for oral application. Also the processing of these mixtures to create powders that are ready for application is not possible without modifications.

In order to compensate for the above-named disadvantages, DE 30 01 357 C2 proposes a laxative in the form of a laxative granulate based on senna fruits, plantago seeds and if required also with plantago seed husks having an improved retarded release action, in which the senna particles of plantago seeds, in particular the slimy substance (mucin) of the plantago seed, are provided in an enveloped or covered status. The composition described therein is an efficient laxative. However, because the composition has the form of a granulate with a granular particle size from about 1 to about 3 mm, that is to say as an asymmetrical aggregate of powder particles, it must be ingested with a large amount of water. In addition, there is also the risk that an inexpert oral intake or application, namely with oral ingestion or application with small water amounts can cause the formation of swollen lumps of granulate, which can in the worst cases lead to an obstruction of the esophagus or even of the gastrointestinal tract.

Laxatives available on the market that are based on the fleaseed (psyllium) or its ingredients often fail to provide a sufficient laxative action, and remain after suspension in water due to a quick onset of the swelling action in a state in which drinking is no longer possible, which means that the aqueous suspension must be ingested immediately after its preparation. After suspension in water, these products will then thicken and form already after a few minutes thick, undrinkable lumps on the surface. In addition, phase separation affecting at least two layers occurs in most of these preparations very quickly, during which process a homogenous distribution of the active components in the mixed preparation is no longer guaranteed. Also, these preparations must be ingested with an abundant amount of water, because otherwise there is the risk that formation of swollen lumps could occur, which can lead in the worst cases to an obstruction of the esophagus or even of the gastrointestinal tract. Another disadvantage of these products is also their bad taste and their external appearance.

That is why the task of the present invention is to provide a composition for use as a laxative which at least substantially prevents the disadvantages illustrated above.

Another task of the present invention is to provide a composition that can be used as a laxative, which on the one hand displays a good laxative action, while on the other hand also enables a simplified application without complications, in particular without the problems or risks illustrated above.

Finally, another task of the present invention is a further development of the laxative disclosed in DE 30 01 357 C2.

The applicant has found to her surprise that a composition or mixture based on plantago seeds and/or plantago seed husks, as well as anthranoid compounds with a laxative or purgative action, in particular sennosides, preferably in the form of plant parts or plant constituents containing anthranoids or sennosides (for instance fruits or leaflets of senna plants), can be processed to prepare a fine powder, which makes it possible to avoid the above-named disadvantages when it is used as a laxative, when these compositions are formulated together with a polygalactomannan or a derivative thereof.

The object of the present invention is thus a pulverized composition, which contains:

(A) plantago seed (Plantaginis ovatae Semen) and/or (A′) plantago seed husks;

(B) at least an anthranoid compound, in particular at least one sennoside (preferably in the form of plant parts or plant constituents containing anthranoids, in particular sennoside); and

(C) in addition, at least a polygalactomannan or a derivative thereof.

Since at least a compound is added containing added anthranoids, in particular sennoside, preferably in the form of parts of plants or plant constituents containing anthranoid or sennoside (for example fruit husks/pods or leaflets of senna plants), a generally complex mixtures is thus provided having different anthranoid compounds or sennosides by the composition according to the invention.

Since the applicant has discovered, to her surprise, that polygalactomannan or derivatives thereof act as a (co)stabilizer with respect to the above-defined pulverized composition when it is added to an aqueous suspension, due to the presence of polygalactomannan-based or polygalactomannan-derived polysaccharides, this means that the pulverized composition according to the present invention improves the suspending or dispersing capability in water, so that an aqueous suspension of the pulverized composition according to the invention will be preserved stable and thus drinkable over a longer period of time, that is to say the suspension will not thicken prematurely, and premature phase separation will also not occur. Moreover, the polygalactomannan also has the somewhat unexpected function of providing additional support for the action of the remaining ingredients, in particular of the plantago seed and possibly also of the plantago husk, because polygalactomannan itself possesses a certain swelling capability;

It therefore has an unexpected synergistic effect, together with the other ingredients of the pulverized composition, and increases in this manner their pharmacological efficacy. Finally, surprisingly enough, polygalactomannan or a derivative thereof supports also the processing characteristics (workability) of the composition according to this invention, which can be processed to form a fine, pourable powder that can be suspended or dispersed in water.

Polygalactomannan or a derivative thereof is added preferably in the form of compounds selected from the guaran group, synonymously also referred to as Guarana gum or Cyamopsis gum), or guar derivatives, in particular fully esterified and/or etherified guar derivatives, especially guar ethers, such as for example carboxymethyl derivatives and hydroxyalkyl derivative and cationically modified products obtained from the reaction of guar powder with monochloroacetic acid, ethylene oxide or propylene oxide and 2,3-epoxypropyl trimethylammonium chloride in the presence of alkali.

Particularly preferred is the addition of polygalactomannan or a derivative thereof in the form of a guar powder and/or guar gum, preferably guar gum. Gum powder and/or guar gum not only acts in a particular manner as a (co)stabilizer of the composition according to the invention in an aqueous suspension, but it also supports the formation of a powder having a particularly fine grain, and it increases due to its own swelling capability in a particular way also the purgative action of the remaining ingredients, thus resulting in a synergistic effect together with the remaining ingredients.

Guar powder is the designation used for a gray-white powder, which has been cultivated and which is obtained by grinding of endosperm, originally in the regions of India and Pakistan, but in the meantime also in other countries, for instance in the South of United States, of guar pod, belonging to the Leguminosae family (Cyamopsis tetragonoloba). The main ingredient of the guar powder is up to 85 weight % of dry guaran substance (guar gum, Cyamopsis gum); secondary ingredients are proteins, lipids and cellulose.

Guaran itself is a polygalactomannan, that is to say a polysaccharide whose linear chain is formed from non-substituted mannose units and mannose units substituted in the C₆ position with a galactose radical in the β-D-(164)- combination. Guaran itself possesses a very high thickening effect (viscosity of a 1.5 weight % aqueous solution of up to 15,000 mPa.s). However, the solutions are clouded to a high degree by insoluble secondary ingredients of the guar powder. The solubility can be significantly improved by derivatization, in particular by etherisation or esterification of guar powder. Further details can be obtained from Römpp Chemistry Lexicon, 10. Edition, Volume 2, 1997, pp. 1622/1623, key words “guar powder” and “guar derivatives”, published by the Georg Thieme Publishing House Stuttgart/New York. The content of the bibliography cited in this source is hereby incorporated by way of reference.

Under guar derivatives are understood in accordance with the invention in particular guar powder derivatives. Derivatization of guaran, the main ingredient of guar powder, can be achieved in various ways as it is a polysaccharide, for example by a full or partial esterification and/or etherification of its hydroxyl groups. Of technical significance are in particular guar esters, in particular carboxymethyl derivatives and hydroxyalkyl derivatives, as well as cationically modified products, which are obtained during the reaction of guar powder with monochloroacetic acid, ethylene oxide or propylene oxide and 2,3-epoxypropyl trimethylammonium chloride in the presence of alkali. The guar derivatives, in particular guar ether, are characterized in contrast to non-modified guar powder by a rapid and improved solubility in water and by an increased solubility of the aqueous solution.

It is particularly advantageous when the pulverized composition in accordance with the invention additionally contains (D) at least a silicic acid or a derivative thereof. The silicic acid supports on the one hand the pourability of the pulverized composition and facilitates its workability enabling to create a fine powder, that is to say it exerts a positive influence on the manufacturing process or speeds up the manufacturing process in that it acts as a flow mediator. On the other hand, silicic acid also has a (co)stabilizing effect in an aqueous suspension of the composition according to the invention, thus providing support for the action of the polygalactomannan or a derivative thereof, in particular by a synergistic interaction with the polygalactomannan or a derivative thereof. Finally, the silicic acid adsorbs moisture which can in some cases be generated during the processing or during storage and thus has the effect of an agent preventing formation of lumps.

A highly disperse, preferably pyrogenous silicic acid is preferred as a time-proven silicic acid, having a SiO₂ content of at least 95% or more, in particular at least 99%. An example of such a suitable silicic acid according to this invention is the product AEROSIL manufactured by the Degussa® Company. This product is manufactured with a hydrolysis of silicon tetrachloride in oxyhydrogen gas flame (2H₂+O2+SiCl₄ 6 SiO₂+4 HCL, flame hydrolysis), which is a highly disperse pyrogenous silicic acid having a SiO₂ content of more than 99.8%. One part of the silicic acid or a derivative thereof can be replaced by maltodextrin.

As far as the sennosides employed in accordance with this invention are concerned, these are generally hydroxyanthracene derivatives, in particular 1,8-dihydroxyantrone derivatives. A complex mixture of various sennosides is generally employed

The sennoside employed according to the invention is in particular selected from the group of the following compounds according to general Formula I: 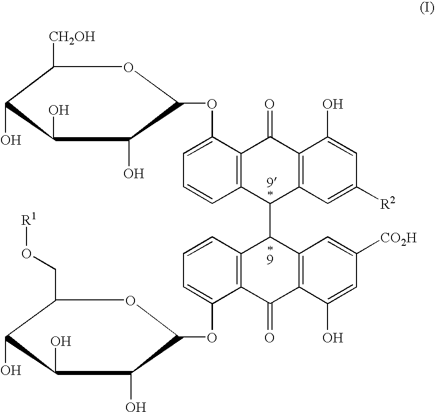
wherein in the general Formula I:

radical R¹ denotes hydrogen or a group —CO-CO₂H,

radical R² denotes a CO₂H or —CH₂OH group, however, with the provision that when R¹ denotes a —CO-CO₂H group, R² denotes a CO₂H group,

which represents a carbon atom indicated by the symbol “*” in position 9 and 9′ of the anthrone-equipped chirality center,

as well as mixtures and/or stereoisomers, in particular enantiomers, and/or diastereoisomers, and/or derivatives of the compounds named above.

The sennoside is preferably selected from the group of the following compounds of the General Formula (I):

| Compound | R¹ | R² | 9-9′ |
|---|---|---|---|
| (IA) | —H | —CO₂H | R*, R* (threo) |
| (IB) | —H | —CO₂H | R*, S* (erythro) |
| (IC) | —H | —CH₂OH | R*, R* (threo) |
| (ID) | —H | —CH₂OH | R*, S* (erythro) |
| (IE) | —CO—CO₂H | —CO₂H | R*, R* (threo) |
| (IF) | —CO—CO₂—H | —CO₂H | R*, S* (erythro) |

as well as mixtures and/or derivatives of the above-named compounds.

It is advantageous to add sennosides in the form of fruits (fructus), in particular for instance as fruit husks, and/or leaflets (folia), preferably fruits or fruit husks of senna plants. Preferred are Tinnevelly senna plants and Alexandrian senna plants, particularly preferred are the Tinnevelly senna plants, preferably in a dried powder that has been finely ground.

Especially preferred is the application of sennosides in the form of fruits (fructus) (in particular for example fruit husks/pods), and/or leaflets (foliae), preferably fruits, of the Tinnevelly senna plant (Senna Angustifolia, Cassia angustifolia).

The Tinnevelly senna plant (Senna angustifolia, Cassia angustifolia) is a shrub having a height of one to two meters, which is at home in the countries bordering on the Red Sea. It is, however, also cultivated in large amounts in the field cultures in India, frequently being alternated with rice. This shrub is grown not only in the vicinity of the city of Tinnevelly (drug designation), but also in the vicinity of Bombay and Madras. The Alexandrian senna plant is at home in North Africa and in the Central Nile region, where it is found as a subshrub growing up to the height of 60 cm, which is often cultivated in Egypt and Sudan. It owes its name to the former export port of Alexandria, and it is also referred to as Khartoum senna. Fruits and leaflets of the senna plants contain a complex sennoside mixture which has a purgative effect. Further details can be obtained from the German Pharmacopoeia, 10. Edition (DAB 10), Deutscher Apotheker Verlag [German Pharmacist Publishing House], Stuttgart, 1991, as well as from the commentary to the DAB, 10. Edition, 1. Shipment, 1993, Part 54, pages 1 through 5, in the dissertation work of the Gerhard-Mercator University in Duisburg “NIR Spectrometry as a Method of Quantitative Analysis of Synthetic and Plant Active Substances in Tablets and Granulates”, published by Dr. Frank Zeyen in the year 2000, as well as in the WHO Monograph (WHO Monographs on Selected Medical Plants) “Folium Sennae” (http:/who.int.medicines/library/trm/medicinalplants/pdf/241to249.pdj).

It is, however, also possible to use other plants or plant ingredients containing anthranoids in accordance with the invention for drugs containing anthrachinone and having a laxative effect, such as for example Rheum palmatum and Rheum officinale (medicinal rhubarb, root), Rhamnus frangula (black alder, bark), Rhamnus pushiana (American alder, bark), Aloe barbadensis, Aloe ferox (Curacao or Cape Aloe, concentrated juice from leaves), Rhamnus catharicus (buckthorn, berries), etc., to name just a few examples.

According to a particular embodiment form of the present invention, the anthranoids or sennosides, in particular particles of the senna plant (fruits or fruit husks and/or leaflets) are present in one of the plantago seeds, in particular in the slimy substance (mucin) of the plantago seed, at least partially or fully enveloped in the composition according to this invention. The composition according to the invention is thus additionally stabilized.

The proportion of the constituent amounts of the individual ingredients is not critical and can vary within a wide range. The content of the plantago seed can vary for example within the range from 30 to 80 weight %, in particular 40 to 70 weight %, preferably 45 to 55 weight %. The content of the plantago seed husks, which can be also present, can also vary within a wide range, namely in the range from 0 to 5 weight %, in particular 1 to 4 weight %, preferably 1.5 to 3 weight %. The content of the sennoside(s) can also vary in a wide range; in general it amounts to 0.05 to 1.0 weight %, in particular 0.08 to 0.6 weight %, preferably 0.1 to 0.5 weight %. The content of the senna plant(s), in particular of the senna fruits or senna fruit husks, can vary within the range from 5 to 25 weight %, in particular 7.5 to 15 weight %, preferably 10 to 13 weight %. The content of the polygalactomannan(s), in particular of guaran and/or guar derivatives (particularly guar powder or guar gum), can vary in the range from 5 to 20 weight %, in particular 7 to 15 weight %, preferably 7.5 to 12 weight %, while 8.0 to 10 weight % is particularly preferred. The content of silicic acid or derivates thereof, which can be also present, can vary within the range from 0 to 5 weight %, in particular 0.01 to 2 weight %, preferably 0.02 to 1 weight %, while 0.05 to 0.1 weight % is particularly preferred. The above-named weight percentages are in each case based on the dry weight of the total composition according to the invention.

In order to improve the workability and/or appearance and/or organoleptic characteristics, it is possible to add to the composition according to the invention also further additives or additional admixtures, for example dyes (for instance natural or organic dyes), flavoring agents, taste enhancers, aromatic substances, (co)stabilizers, fillers, processing characteristics enhancing agents, sweeteners and the like, as well as mixtures of the above-named compounds.

A preferred pulverized composition according to the invention has the following formulation, wherein the amount data are in each case based on the dry weight of the total composition:

| | Parts by Weight |
|---|---|
| ^{!}(A) Plantago seed: | 30 to 80, in particular 40 to 70, preferably 45 to 55. |
| ^{!}(A′) Plantago seed | 0 to 5, in particular 1 to 4, preferably 1.5 to 3 |
| husks: | |
| ^{!}(B) Tinnevelly | 5 to 25, in particular 7.5 to 15, preferably 10 to 13 |
| senna fruits: | (corresponding to 0.05 to 1.0 of parts by weight of |
| | sennosides, in particular 0.08 to 0.6, preferably 0.1 |
| | to 0.5 parts by weight) |
| ^{!}(C) Guar powder | 5 to 20, in particular 7 to 15, preferably 7.5 to 12, |
| and/or guar gum: | particularly preferred is 8.0 to 10 |
| ^{!}(D) Silicic acid: | 0 to 5, in particular 0.1 to 2, preferably 0.02 to 1, |
| | particularly preferred is 0.05 to 1 |
| ^{!}(E) Taste enhances | 0 to 60, in particular 20 to 50, preferably 35 to 45 |
| and dyes: | |

The workability of the individual active ingredients needed to achieve a stable, fine, homogenous powder mixtures is, based on the research of the applicant, possible only thanks to the integration of the polygalactomannan or derivatives thereof according to the invention, in particular of the guar powder and/or guar rubber, which can be also present together with fine particles of silicic acid; wherein the polygalactomannan or a derivative thereof has an additional stabilizing effect with respect to the aqueous suspension of the powder composition according to the invention, which clearly facilitates oral intake.

The bulk of the components of the composition according to the invention, in particular more than 60 weight %, in particular more than 70 weight %, preferably more than 75 weight %, displays grain sizes in the range from 125 to 250 Φm.

The composition according to the invention is in general characterized by the following distribution of grain sizes (sieve analysis according to DIN ISO 3310-1):

more than 95 weight %, in particular more than 99 weight %, finer than 500 Φm;

more than 70 weight %, in particular more than 75 weight %, finer than 250 Φm;

more than 10 weight %, in particular more than 15 weight %, finer than 125 Φm.

In an advantageous manner, the individual constituent parts of the composition according to the invention are provided in an intimate, preferably homogenous mixture, that is to say the individual constituent parts are intimately, preferably homogenously, processed or blended with each other.

The composition according to the invention is extraordinarily capable of swelling. Stimulation of the bowel function is in this manner achieved through physical excitation. The swelling number (according to DAB 8, German Pharmacopoeia, 8. Edition, 1978, Official Edition, The German Pharmacist Publishing House, Stuttgart/Covi Verlag GmbH Frankfurt, starting from page 24) is in particular at least 5.0, in particular preferably 6.0, preferably at least 7.0, while 7.5 is particularly preferred, and at least 8.0 and more is especially particularly preferred.

As described above, the particles of the ground senna fruits can be also provided with a protective layer by being enveloped by slimy substances of the plantago seed ground product. With this protective cover, the sennosides are substantially protected from undesirable changes and their release is delayed, so that an even longer lasting, retarded and milder action can be achieved.

Experiments conducted by the applicant with the composition according to the invention, prove a delayed release of the sennosides from the laxative according to the invention, wherein the retarded release of the sennosides, however, does not lead under any circumstances to a full blocking of the active substances, but rather only to a desirable complete releasing, which takes place practically in its entirety over a longer period of time. This makes it possible for the sennosides to fully exert their effect.

The pulverized composition according to the invention is thus eminently suitable for use as a laxative (purgative means).

Due to the fact that the composition according to the invention is provided in the form of a finely ground powder, it can be particularly well worked into water or dispersed in it and it remains easily drinkable for a long time after being stirred in water, that is to say in contrast to products based the existing status of technology, a rapid separation of phases will not take place. In contrast, gelatinized products are formed based on the existing status of technology already after a few minutes, so that a thick, undrinkable mass is formed on the surface.

In addition, the composition according to the invention also possesses a natural appearance as well as a natural taste.

The composition according to the invention displays a good purgative action and is, as a result of gentle application when used according to indications, substantially free of undesirable side effects, in particular thanks to a good compatibility. It is thus suitable for support of bowel functions, in particular also with hemorrhoid or fissure patients, and even after a surgical intervention. In the same manner, the composition according to the invention can be also employed for clinical regulation of immobilized patients, that is to say in particular with patients who are confined to bed over a longer period of time, and it can be without reservations also administered during pregnancy. The composition according to the invention can thus be used with all illnesses when easy defecation with a soft stool is desirable (for instance with anal fissures, with hemorrhoids, after rectal-anal surgical interventions, for cleansing of bowels prior to X-ray examinations, and before and after surgical interventions in the abdominal cavity region or in the gastrointestinal tract, with constipation, etc.).

The composition according to the invention is also suitable for example for treatment of patients with drug-induced constipation, for instance during treatment with Loperamid® which is frequently employed. A study by von Ewe et al. (Pharmacology 1993, 47, Suppl. 1, pages 242 through 248) documented a shortened intestinal passage when Loperamid® was used simultaneously with preparations containing sennosides. Surprisingly enough, it was found that among other reasons due to the “fluid” administration of the composition according to this invention in the form of an aqueous suspension and due to the fine particles, this effect is better than with conventional preparations because the fluid application is still possible in the form of an aqueous suspension having the finest particles also with a transient restriction of the passage from the stomach into the intestines for solid, fine particles within the context of a “fluid” preparation (aqueous suspension). Consequently, the composition according to the invention is suitable also for facilitation of the intestinal passage and defecation, in particular for mitigation of side effects due to drug therapy causing constipation—albeit only as an undesirable side effect.

Toxicity studies have proven that the composition according to the invention does not lead to acute toxicity, nor does it lead with application according to instructions to a chronic toxicity risk. Also no choleritic effect of any kind was observed. Further, no incompatibilities of any kind were observed either.

The pulverized composition according to the invention is thus eminently suitable for use as an effective laxative, which with oral ingestion according to instruction leads to no undesirable side effects, such as in particular obstruction of the esophagus or of the gastrointestinal tract.

The composition according to the invention thus combines in an efficient manner the physical action of the plantago seed based on the swelling characteristics with the pharmacologically stimulating mode of action of sennosides, the ingredients present in the senna fruits, so as to achieve an improved total action in this manner.

Due to the pulverized character, the composition according to the invention is used for oral application as a suspension in water. The ease with which the composition can be suspended and the consequent stabilization of a suspension prepared in this manner are ensured by the polygalactomannan-based or polygalactomannan-derived polysaccharide, in particular in the form of a guar powder and/or guar gum, when required also together with silicic acid, so that oral intake occurs automatically with sufficient amounts of water, as the composition is supplied in the form of an aqueous suspension.

It is advantageous when the composition according to the invention is already provided for this purpose packaged in the form of single-dose packages whose respective amounts preferably correspond to a single dose of the composition according to the invention (for example 5 to 10 g of the composition according to the invention per package or similar such amounts of the composition, which correspond to 5 to 20 mg of sennosides). In order to provide protection to a sufficient extent for the composition according to the invention, the single-dose packages are preferably constructed so that they are at least substantially hermetically sealed and/or at least substantially waterproof (for example by heat-sealing or gluing). It is advantageous when the single-dose packages of the composition according to the invention are also protected from the influence of light, in particular from ultraviolet light rays.

The composition according to the invention is generally ingested orally, in particular in amounts from 1 to 50 g, in particular 1 to 20 g, preferably 5 to 10 g of the composition per day. The daily dose of 60 mg, preferably 30 mg of sennoside(s), should be preferably not exceeded in this case. On the other hand, in order to achieve an adequate purgative action, the composition according to the invention should be administered in amounts corresponding to a daily does of sennoside(s) of at least 5 mg, in particular at least 10 mg, preferably at least 15 mg. It can be in this case advantageous to divide the total amount into several single doses, for example into two to three single doses, each from 5 to 25 g, in particular 5 to 20 g, preferably 5 to 10 g of the composition per a single dose, or with 5 to 10 mg, in particular 5 to 10 mg of sennoside(s).

The oral intake should be combined with a sufficient supply of water to guarantee on the one hand an efficient action (swelling), and on the other hand also to avoid undesirable side effects (such as for instance obstruction of the esophagus). This can be generally ensured with oral intake in the form of an aqueous suspension of the pulverized composition according to the invention, because otherwise the composition according to the invention cannot be ingested in its finely pulverized form. In general, at least 40 ml of water should be provided with oral intake per 1 g of the composition according to the invention, wherein suspension of the composition according to the invention is induced in water prior to oral intake, which is followed by application in the form of an aqueous suspension.

The manufacturing of the pulverized form of the composition according to the invention can be realized according to a method that is per se known. The manufacturing takes place generally according to a conventional manufacturing procedure for powder compositions. This can be achieved for example so that the individual constituent parts are first finely ground, (when applicable after drying), wherein the degree of grinding can be targeted according to the desired granulometry of the end composition so that consequently, an intimate homogenous mixture of the individual constituent parts is prepared in the desired amount relationships. However, grinding of the individual constituent parts can alternatively also take place only in the mixed status.

The present invention further also relates to the use of the composition according to the invention for the manufacturing of a medicine or of a pharmaceutical composition for promotion or facilitation and/or regulation of intestinal evacuation or of bowel movements. The present invention thus relates to the use of the composition according to the invention for prophylactic and/or curative (therapeutic) treatment of the above-named illness conditions, during which easy defecation with a soft stool is desirable.

Finally, the present invention also relates to a therapeutic procedure for promotion and/or regulation of intestinal evacuation or bowel movements (for example for treatment of obstruction or constipation or other diseases, during which easy defecation with a soft stool is desirable as described above), wherein the above described composition according to the invention is administered in therapeutically effective amounts, in general with oral application, preferably with an oral supply of an aqueous suspension of a pulverized composition according to the present invention.

Other refinements, modifications and variations, as well as the advantages of the present invention can be easily recognized and realized by a person in the art upon reading of the description, without deviating from the framework of the present invention. The following exemplified embodiments serve solely to illustrate the present invention, while the invention is not limited by the embodiments.

### EXEMPLIFIED EMBODIMENTS

### Example 1

A composition according to the invention and two comparison products according to the existing state of technology were transferred to an aqueous suspension. 6 g of the compositions were mixed for this purpose in approximately 240 ml of tap water.

The composition according to the invention is a finely ground powder (more than 70 weight % of the particles have a median diameter in the range from 125 to 250 μm), which contains 52 weight % of plantago seed, 2.2 weight % of plantago seed husks, 12.3 weight % of Tinnevelly senna fruits (corresponding to approximately 0.3 weight % of sennosides), 8.3 weight % of guar rubber, 0.07 weight % of fine particles of silicic acid, 0.03 weight % of maltodextrin and 25.1 weight % of other ingredients (natural aromatic substances, dyes, taste enhancers and sweeteners).

The first comparative composition is a composition containing 52 weight % of plantago seed, 2.2 weight % of plantago seed husks, 10 to 13.2 weight % of Tinnevelly senna fruits (corresponding approximately to 0.3 weight % of sennosides) and 32.6 to 35.8 weight % of other ingredients (talc, gum Arabic, iron oxides, paraffin, aromatic substances, saccharose). It is, however, free of guar gum and silicic acid.

The second comparative composition is a trade product based on Indian fleaseed (psyllium), which additionally contains also other ingredients (among other things natural and synthetic dyes, taste enhances, aromatic substances and sweeteners, citric acid, iron oxide, calcium), but which is free of sennosides and which also does not contain any guar gum and no silicic acid.

After suspension of three compositions in water, only the composition according to the invention remained stable in the water for more than 15 minutes, while both comparison suspensions displayed phase separation already after a few minutes (sedimentation of solid substances). The second comparative composition, which was also inferior due to its bad taste and artificial external appearance, thickened into a jelly-like substance already after 15 minutes and formed a thick, undrinkable mass on the surface, while the suspension of the composition according to the invention remained drinkable even after one hour.

There were significant differences between the three substances also in their laxative effect: while the second comparative composition based Indian fleeseed (psyllium) displayed only a moderate purgative action, which is not always sufficient in serious cases of constipation, the second comparative composition clearly displayed a better purgative action based on the combination of the physical action of the plantago seed husks due on the one hand to swelling, and on the other hand thanks to the pharmacologically stimulating action of the sennosides. This combined action is further increased with the composition according to the invention based on the synergistic manner of the action of the guar gum.

### Example 2

A composition according to the invention is prepared for instance as follows:

Starting Mixture

| Ingredients | g/Dose Unit | g/Charge |
|---|---|---|
| Plantago seed | 3.12 | 512,600 |
| Plantago seed husks | 0.13 | 23,400 |
| Tinnevelly senna fruits | 0.74 | 133,200 |
| Natural dyes and taste enhancers | 1.5058 | 271,044 |
| Guar gum | 0.5 | 90,000 |
| Aerosil ® | 0.0042 | 756 |
| | Γ 6.00 g | Γ 1,080.000 g |

Sieving and Mixing

The individual ingredients—provided in each case in finely ground form—were introduced by means of a vacuum into a double-cone mixture (a mixer that is tapered on both sides) which had a holding capacity of 2,500.

Lumps or larger constituent parts were separated with a 3 mm sieve during the loading of the mixer. The supplied materials were then mixed for a period of 30 minutes with the speed of 16 revolutions/minute. In addition, the homogenized, mixed product was filled into two polypropylene bags placed below (Bigbags), which were equipped with inner polyethylene bags. The result was a mixture or composition according to the invention.

Sachet Filling and Packing Process:

The pulverized product, manufactured in advance, was supplied by means of a vacuum to a device for simultaneous filling of four sachets at a time (single-dosage packages). Each of the individual sachets was charged with a dose of 6 g, determined ahead of time, via a screw dosing apparatus. The charge No. and the expiration date were imprinted on the flap of the sachet. In addition, the sachets were sealed by heat-sealing and heat-baking so that the content was packaged in a hermetically sealed and waterproof state. The charged and sealed sachets were then stocked in pairs for a total of 20 sachets and transported to a carton processing or packing machine where they were packed together with a packing ticket that was enclosed in the package, and the No. of each charge and the expiration date were printed on the carton. Thereafter, the correct weight of the units was ascertained and units displaying fluctuations were discarded when applicable. A predetermined number of the loaded cartons was then supplied for further packing in large packages.

1-30. (Cancelled)

31. A pulverized composition, comprising:
plantago seed matter selected from the group consisting of seeds and seed husks of Plantaginis ovatae Semen;
an anthranoid compound; and
a polygalactomannan or a derivative thereof.

32. The composition according to claim 31, wherein the anthranoid compound is a sennoside.

33. The composition according to claim 31, wherein the anthranoid compound is provided in the form of anthranoid-containing plant parts or constituent parts of plants.

34. The composition according to claim 31, further comprising a compound selected from the group consisting of silicic acid and derivatives thereof.

35. The composition according to claim 34, wherein the silicic acid is a highly disperse pyrogenous silicic acid comprising at least 95% SiO₂.

36. The composition according to claim 34, wherein a portion of said silicic acid is replaced by maltodextrin.

37. The composition according to claim 31, wherein the polygalactomannan or a derivative thereof is selected from a group comprising guaran (guar rubber, Cyamopsis gum) and guar derivatives.

38. The composition according to claim 37, wherein said quar is derived by a process selected from the group consisting of esterification, etherification, carboxymethylation, hydroxyalkylation, and cationization.

39. The composition according to claim 38, wherein said cationization comprises reaction with a compound selected from the group consisting of monochloroacetic acid, ethylene oxide or propylene oxide, and 2,3-epoxypropyl trimethylammonium chloride.

40. The composition according to claim 31, wherein the polygalactomannan comprises guar powder or guar rubber.

41. The composition according to claim 31, wherein the composition contains a plurality of anthranoid compounds.

42. The composition according to claim 31, wherein the anthranoid compound is a 1,8-dihydroxanthrone derivative.

43. The composition according to claim 31, wherein the anthranoid compound is selected from the group consisting of compounds having General Formula (I) stereoisomers thereof, and derivatives thereof, wherein 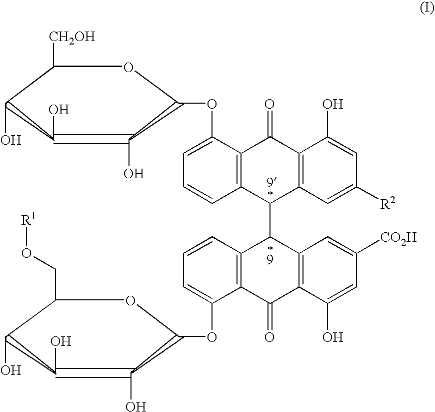
the radical R¹ represents hydrogen or a group —CO-CO₂H,
the radical R² denotes a CO₂H or —CH₂OH group, however, with the provision that when R¹ denotes a CO-CO₂H group, R² denotes a CO₂H group, and wherein
an asterisk represents a 9- or 9′-carbon atom of the anthrone-derived chiral center.

44. The composition according to claim 42, wherein the anthranoid compound is selected from the group of the following compounds according to General Formula (I):

| Compound | R¹ | R² | 9-9′ |
|---|---|---|---|
| (IA) | —H | —CO₂H | R*, R* (threo) |
| (IB) | —H | —CO₂H | R*, S* (erythro) |
| (IC) | —H | —CH₂OH | R*, R* (threo) |
| (ID) | —H | —CH₂OH | R*, S* (erythro) |
| (IE) | —CO—CO₂H | —CO₂H | R*, R* (threo) |
| (IF) | —CO—CO₂—H | —CO₂H | R*, S* (erythro) |

45. The composition according to claim 31, wherein the anthranoid compound is added in the form of fruits (fructus) and/or leaflets (folia) of senna plants.

46. The composition according to claim 44, wherein the senna plants are selected from the group consisting of Alexandrian senna plants and Tinnevelly senna plants.

47. The composition according to claim 31, wherein the anthranoid compounds comprise dried and finely powdered fruits and/or leaflets of Tinnevelly senna plant (Senna angustifolia, Cassia angustifolia).

48. The composition according to claim 31, further comprising additional additives.

49. The composition according to claim 47, wherein said additive is selected from the group consisting of dyes, flavoring agents, taste enhancers, aromatic substances, stabilizers, (co)stabilizers, fillers, processing characteristics enhancing agents, as well as mixtures thereof.

50. The composition according to claim 31, wherein more than 99 weight % of said composition has a grain size finer than 500 micrometers.

51. The composition according to claim 31, wherein more than 95 weight % of said composition has a grain size finer than 500 micrometers.

52. The composition according to claim 31, wherein more than 70 weight % of said composition has a grain size finer than 250 micrometers.

53. The composition according to claim 31, wherein more than 75 weight % of said composition has a grain size finer than 250 micrometers.

54. The composition according to claim 31, wherein more than 15 weight % of said composition has a grain size finer than 125 micrometers.

55. The composition according to claim 31, wherein more than 10 weight % of said composition has a grain size finer than 125 micrometers.

56. The composition according to claim 31, wherein more than 60 weight % of the composition displays grain sizes in the range from 125 to 250 m.

57. A pulverized composition comprising:
30 to 80 parts by weight plantago seed;
0 to 5 parts by weight plantago seed husks;
5 to 25 parts by weight Tinnevelly senna fruits;
5 to 20 parts by weight Guar powder and/or guar gum;
0 to 5 parts by weight silicic acid;
0 to 60 parts by weight Taste enhancers and dyes.

58. The composition according to claim 57, wherein more than 99 weight % of said composition has a grain size finer than 500 micrometers.

59. The composition according to claim 57, wherein more than 95 weight % of said composition has a grain size finer than 500 micrometers.

60. The composition according to claim 57, wherein more than 70 weight % of said composition has a grain size finer than 250 micrometers.

61. The composition according to claim 57, wherein more than 75 weight % of said composition has a grain size finer than 250 micrometers.

62. The composition according to claim 57, wherein more than 15 weight % of said composition has a grain size finer than 125 micrometers.

63. The composition according to claim 57, wherein more than 10 weight % of said composition has a grain size finer than 125 micrometers.

64. The composition according to claim 57, wherein more than 60 weight % of the composition displays grain sizes in the range from 125 to 250 m.

65. A method of using the composition of claim 31 comprising administering said compound to a human or other mammal.

66. The method of use, according to claim 65 wherein said administration is per oral.

67. The method of claim 65, wherein said composition is administered as an aqueous suspension in daily doses of a total of 1 to 50 g, with respect to the dry weight of the composition.
